(19) United States
(12) Patent Application Publication
(10) Pub. No.: US 2026/0273213 A1
(43) Pub. Date: Sep. 17, 2026

(54) TRACHEAL CANNULA AND USE OF A TRACHEAL CANNULA

(71) Applicant: ANDREAS FAHL MEDIZINTECHNIK-VERTRIEB GMBH, Köln (DE)

(72) Inventors: Andreas Fahl, Köln (DE); Thomas Bender, Much (DE)

(73) Assignee: ANDREAS FAHL MEDIZINTECHNIK-VERTRIEB GMBH, Köln (DE)

(21) Appl. No.: 19/558,866

(22) Filed: Mar. 6, 2026

Related U.S. Application Data
(63) Continuation of application No. PCT/EP2024/074908, filed on Sep. 6, 2024.

(30) Foreign Application Priority Data
Sep. 7, 2023 (DE) 102023124087.0

(51) Int. Cl. A61M 16/04

(52) U.S. Cl. CPC A61M 16/0465; A61M 16/0429; A61M 16/0431; A61M 16/0434; A61M 16/0488

(57) ABSTRACT

A tracheal cannula is provided, at least including a cannula tube and a cannula shield. The cannula tube includes a distal end portion, to which the cannula shield is assigned, and a tracheal end portion which is able to be arranged in a trachea. The cannula tube includes a cannula lumen which is continuous from a distal end to a tracheal end and which has a constant internal diameter. A wall at least of the tracheal end portion includes a first wall thickness, which is contiguous with the distal end portion, and a second wall thickness at the tracheal end, wherein the first wall thickness is greater than the second wall thickness. The wall thickness of the tracheal end portion continuously reduces towards the tracheal end.

### CROSS-REFERENCE TO RELATED APPLICATIONS

This Application is a continuation of International Application No. PCT/EP2024/074908, filed on Sep. 6, 2024, which in turn claims priority to German Application No. 1020231240870, filed on Sep. 7, 2023, the entire contents of both incorporated herein by reference in their entirety.

### TECHNICAL FIELD

The present disclosure relates to a tracheal cannula and a use of a tracheal cannula.

### BACKGROUND

Tracheal cannulas are generally known from the prior art. DE 20 2017 002 009 U1 thus discloses a system comprising a tracheal cannula and an insertion aid, wherein the insertion aid and the tracheal cannula are able to be coupled in a form-fitting manner.

U.S. Pat. No. 10,864,339 B2 discloses a breathing tube, the construction of which provides a constant internal diameter. DE 2 155 088 A1 discloses an artificial trachea for inserting into the natural trachea to support breathing with a cannula.

A disadvantage in the prior art is that the insertion of the tracheal cannula usually takes place in a traumatic manner. Known tracheal cannulas thus have a shoulder between a tip of an obturator and an end portion of the tracheal cannula. Furthermore disadvantageously, the wall and/or tubes applied to the wall are obstructive during the insertion of the tracheal cannula. Cannula shields which are not able to be adapted sufficiently to the anatomy of the user are furthermore disadvantageous.

### SUMMARY

It is an object of the present disclosure to provide an improved tracheal cannula.

The problem is solved according to the present disclosure by a tracheal cannula at least including a cannula tube and a cannula shield, wherein the cannula tube includes a distal end portion to which the cannula shield is assigned, and a tracheal end portion, which can be arranged in a trachea, wherein a first external diameter at the cannula shield is assigned to the cannula tube, wherein a second external diameter is assigned to the cannula tube at a tracheal end, wherein the cannula tube includes a cannula lumen which is continuous from a distal end to a tracheal end and has a constant internal diameter, wherein a wall at least of the tracheal end portion includes a first wall thickness, which is contiguous with the distal end portion, and a second wall thickness which is contiguous at the tracheal end, wherein the first wall thickness of the wall at the site of the first external diameter is greater than a second wall thickness at the site of the second external diameter, wherein the first wall thickness is greater than the second wall thickness, and the wall thickness of the tracheal end portion continuously reduces towards the tracheal end.

A tracheal cannula at least including a cannula tube and a cannula shield is provided. The cannula tube includes a distal end portion to which the cannula shield is assigned, and a tracheal end portion which can be arranged in a trachea. The cannula tube includes a cannula lumen which is continuous from a distal end to a tracheal end and has a constant internal diameter. A wall at least of the tracheal end portion includes a first wall thickness, which is contiguous with the distal end portion, and a second wall thickness at the tracheal end, wherein the first wall thickness is greater than the second wall thickness. The wall thickness of the tracheal end portion reduces towards the tracheal end over the entire length of the tracheal end portion. The wall thickness of the tracheal end portion reduces towards the tracheal end continuously.

In a configuration, provision is made that the cannula tube includes a wall which includes at least a first tube guide in which at least a first tube is arranged, wherein at least the first tube guide is formed at least partially as a groove in the wall, which includes at least partially a depth which is greater than or at least approximately equal to an external diameter of the first tube.

When the term “approximately” is used within the present disclosure in connection with values or ranges of values, this is thus to be understood as a tolerance range which the specialist in this field considers to be usual; such as, without limitation, a tolerance range of ±20%, in some embodiments, ±10%, further in other embodiments, ±5% is provided.

In so far as various ranges of values, for example preferred and further preferred ranges of values are indicated in the present disclosure, the lower limits and the upper limits of the various ranges of values are able to be combined with one another. The term “depth” of the first and/or second tube guide describes the distance from the edge at which the first and/or second tube guide is contiguous with the peripheral surface of the cannula tube, to the deepest point of the first and/or second tube guide or respectively a groove base of the first and/or second tube guide.

The term “substantially” indicates a tolerance range which is acceptable for the specialist from economic and technical points of view, so that the corresponding feature is still to be recognized or realized as such.

Exemplary enumerations are not to be regarded as conclusive in the sense of the present disclosure, but can be supplemented within the scope of general technical knowledge.

The terms “continuous” and “constant” in the sense of the present disclosure is to be understood as a continuity for example of a surface, which is generally uninterrupted. For example, a continuous reduction of the wall thickness is to be understood to mean that the wall thickness reduces more and more over the longitudinal extent of the cannula tube, such as of the tracheal end portion. No substantial interruption of the reduction takes place over the longitudinal extent. “Continuous” and “constant” is to be understood to mean that no gap or offset can be detected visually and/or in a tactile manner in a surface or in a cross-section of a surface. For example, substantially no joint or gap is present between the peripheral surface of the cannula tube and the first and/or second tube. For example, the wall of the tracheal end portion, which continuously reduces towards the tracheal end, includes no offset or gap, such as over its entire length.

The term “cross-section” is to be understood as a sectional view which is perpendicular to a longitudinal extent of the cut part. As such, a vector of the viewing level of the cross-section is parallel to the vector of the longitudinal extent at the interface.

The term “flush” is understood in the sense of the present disclosure to mean that two parts are contiguous with one another directly and their surfaces are substantially complementary to one another. For example, the tube can be configured flush with the peripheral surface of the cannula tube, wherein at least a portion of the surface of the tube and a portion of the peripheral surface are arranged without a gap with respect to one another. In embodiments, parts which are arranged in a flush manner have the appearance that their surfaces at least partially complement one another.

In an embodiment, provision is made that the cannula tube includes a tracheal end portion and a distal end portion. The distal end portion is formed in such a way that it has a superior face and an inferior face arranged lying opposite thereto. The cannula shield has a cannula tube cutout into which the distal end portion of the cannula tube is able to be arranged. The cannula shield has a rectilinear first portion, delimiting the cannula tube cutout, which first portion is assigned to the superior face of the distal end portion, and a rectilinear second portion, delimiting the cannula tube cutout, which second portion is assigned to the inferior face of the distal end portion. The cannula shield is arranged in a tiltable manner about a medial-lateral axis by means of a pivot bearing at the distal end portion.

A medial-lateral axis is defined as the axis which, on conventional use of the tracheal cannula, stands substantially perpendicularly on the median plane or respectively mirror plane of the body symmetry of a user. The medial-lateral axis may extend from medial in the lateral direction.

The tracheal cannula includes a cannula tube. The cannula tube includes a distal first end and a proximal second end.

When directional information is used within the description of the present disclosure, this is to be understood in relation to the conventional usage of the tracheal cannula. Directional designations are used as is conventional in anatomy. The term “distal” is understood in the sense of the present disclosure in relation to a feature of the device according to the present disclosure to mean an arrangement or respectively a use thereof distant or also facing away from or lying opposite the tracheal cannula, more generally a skin surface of a person who is carrying the device according to the present disclosure. Distal is to be understood to be a direction away from the patient's body. The term “proximal” is understood in the sense of the present disclosure in relation to a feature of the device according to the present disclosure to mean an arrangement or use thereof close to or also facing a skin surface of a person who is carrying such fixing means for the device according to the present disclosure. Proximal is to be understood to mean a direction towards the patient's body. “Lateral” is to be understood to mean a direction laterally to the right and/or left from a median plane or respectively a median line. “Superior” is to be understood to mean a relative directional indication along or parallel to the longitudinal axis of the body above a reference. “Inferior” is to be understood to mean a relative directional indication along or parallel to the longitudinal axis of the body below a reference. “Tracheal” is to be understood to mean a relative directional indication which, in conventional use, is facing the trachea.

The cannula tube includes a cannula lumen which is continuous from the distal first end to the tracheal second end. The cannula lumen includes an internal diameter. In a configuration, provision is made that the cannula lumen, continuous from the distal end to the tracheal end, includes a constant internal diameter over an entire longitudinal extent, such as over a longitudinal extent of the tracheal end portion.

In the sense of the present disclosure, a constant internal diameter is to be understood to mean that the internal diameter is approximately identical substantially over the entire longitudinal extent of the tracheal cannula or at least of the tracheal end portion. In embodiments, some tolerance deviations of approximately 0.5 mm to approximately 2.5 mm, and in other embodiments of, approximately 1 mm, are also to be understood as “constant”.

The cannula tube includes a wall. The wall surrounds the cannula lumen. The wall includes a peripheral surface. The peripheral surface is the outer surface of the wall of the cannula tube. In a configuration, the peripheral surface or respectively the wall is configured in a screened, fenestrated or nonfenestrated manner. The peripheral surface has no gap in longitudinal direction of the cannula tube. Further, the peripheral surface has a continuous course in longitudinal direction of the cannula tube.

In a configuration, provision is made that an outer circumference of the tracheal cannula is at least partially substantially oval in a cross-section perpendicularly to an extent of the cannula tube.

The wall includes a first tube guide. In a configuration, provision is made that the tracheal cannula includes at least the first tube guide, which extends from a distal side of the cannula shield. In other embodiments, the at least the first tube guide extends from a distal side of the distal end portion of the cannula tube, at least up to a cuff which is assigned to the tracheal end. The tube guide extends up to the tracheal end of the cannula tube. The first tube guide is configured to receive a first tube. In a configuration, provision is made that the first tube guide is arranged laterally in the wall. The first tube guide is configured as a recess in the wall. The tube recess extends in a longitudinal direction of the cannula tube, of the tracheal end portion. In a configuration, provision is made that the at least one first tube guide is configured at least partially as a groove in the peripheral surface of the wall. Provision is made that the first tube guide includes a constant depth over the length of the groove or a constant material thickness from the groove base to the inner wall of the cannula. In a further configuration, provision is made that the depth varies from distal in the tracheal direction. The depth becomes constantly less from distal in the lateral direction.

In a configuration, provision is made that the wall of the cannula tube, at least of the tracheal end portion, has a material buildup laterally at least on one side in a cross-section perpendicularly to the longitudinal extent of the cannula tube. The material buildup thickens the cannula tube at least on one side, such as, without limitation, laterally. The cannula tube includes a material buildup at least at the tracheal end portion at least partially in a cross-section perpendicularly to the longitudinal extent of the cannula tube on both lateral sides. No material buildup is arranged in the region of the tracheal end of the cannula tube or respectively at the tracheal end of the cannula tube. A thickened region of the cannula tube includes a material buildup. A thickened region of the wall or respectively the material buildup extends on a lateral side of the cannula tube, such as on the tracheal end portion, in longitudinal direction of the cannula tube. In a configuration, a wall thickness of the thickened region, or respectively of the region of the material buildup, reduces in longitudinal direction of the cannula tube. The wall thickness of the thickened region, or respectively of the region of the lateral buildup, continuously reduces in longitudinal direction of the cannula tube. The wall thickness of the thickened region, or respectively of the region of the lateral material buildup, reduces in longitudinal direction of the cannula tube to the same extent as the wall thickness of the inferior region and/or of the superior region. The wall thickness of the thickened region, or respectively of the region of the lateral material buildup reduces more quickly in longitudinal direction of the cannula tube than the wall thickness of the inferior region and/or superior region. In a configuration, provision is made that at least the first tube guide is formed in a thickened region of the wall or respectively in the material buildup. Further, provision is made that at least the second tube guide is formed in a thickened region of the wall or respectively in the material buildup. A tube guide is formed as a groove in a material buildup.

The first tube guide is rectangular, oval, lenticular, curved or circular in a cross-section of the cannula tube. In a configuration, the first tube guide into a cross-section resembles an oval segment. In a configuration, the first tube guide is configured in a u-shaped manner in a cross-section perpendicularly to the longitudinal extent of the cannula tube.

In a configuration, provision is made that the first tube guide is configured in a curved or circular-segment-shaped manner in a cross-section perpendicularly to the longitudinal extent of the cannula tube. The cross-section of the first tube guide is adapted to a cross-section of a first tube. The first tube guide is adapted to receive the first tube. The first tube, inserted into the first tube guide, forms or supplements the peripheral surface. The first tube, inserted into the first tube guide, forms or supplements the peripheral surface in such a way that the latter is formed in a constant manner. The first tube guide is configured in such a way that the first tube is formed in the latter in a flush manner with the peripheral surface of the cannula tube.

The wall includes a second tube guide. The second tube guide is configured to receive a second tube. In a configuration, provision is made that the tracheal cannula includes at least a second tube guide, which extends from a distal side of the cannula shield at least up to the cuff and in which a second tube is guided. In a configuration, provision is made that the second tube guide extends up to the second tracheal end of the cannula tube. In a configuration, provision is made that the second tube guide is arranged laterally in the wall. The second tube guide is arranged laterally lying opposite the first tube guide. The second tube guide is configured as a recess in the wall. In a configuration, provision is made that the second tube guide is configured in a tube-shaped manner. In a configuration, provision is made that at least the second tube guide is formed at least partially as a groove in the peripheral surface of the wall. Provision is made that the second tube guide includes a constant groove depth over the length of the groove or a constant material thickness from the groove base to the inner wall of the cannula. In a further configuration, provision is made that the groove depth varies from distal in the tracheal direction. The groove depth from distal in the tracheal direction becomes constantly less. The groove depth becomes constantly less from distal in the tracheal direction through the continuous reduction of the wall thickness of the thickened region, or respectively of the region of the lateral material buildup, in longitudinal direction of the cannula tube.

The second tube guide is rectangular, oval, lenticular, curved or circular in a cross-section of the cannula tube. In a configuration, the second tube guide into a cross-section resembles an oval segment. In a configuration, the second tube guide is configured in a u-shaped manner in a cross-section perpendicularly to the longitudinal extent of the cannula tube. In a configuration, provision is made that the first tube guide is configured in a curved or circular-segment-shaped manner in a cross-section perpendicularly to the longitudinal extent of the cannula tube. The cross-section of the second tube guide is adapted to a cross-section of a second tube. The second tube guide is adapted to receive the second tube. The second tube, inserted into the second tube guide, forms or supplements the peripheral surface. The second tube, inserted into the second tube guide, forms or supplements the peripheral surface in such a way that the latter is configured in a substantially constant manner. The second tube guide is configured in such a way that the second tube is formed therein flush with the peripheral surface of the cannula tube.

In an embodiment, the first and/or the second tube guide are configured in such a way that it becomes flatter towards the tracheal end by a narrowing of the cannula tube over the length of the cannula tube. In an embodiment, provision is made that the first and/or second tube has approximately the same external diameter, internal diameter and/or the same cross-section over its length or over a portion. In an embodiment, provision is made that the first and/or second tube changes the external diameter, the internal diameter and/or the cross-section over its length. The external diameter, the internal diameter and/or the cross-section of the first and/or second tube is adapted over its length to the first and/or second tube guide. In an embodiment, provision is made that the first and/or second tube in a portion contiguous with the distal end portion is received substantially entirely by the first and/or second tube recess in the tracheal end portion, or respectively terminates substantially in a flush manner with the peripheral surface. In an embodiment, provision is made that the first and/or second tube in the tracheal end portion is received substantially entirely by the first and/or second tube recess its entire length or respectively terminates in a substantially flush manner with the peripheral surface. It is advantageous in the complete receiving of the first and/or second tube over the entire length of the tube recess in the tracheal tube that a traumatising of the tissue is avoided on inserting and removing of the tracheal cannula into and from the trachea. In an embodiment, provision is made that the first and/or second tube in extension direction of the first and/or second tube guide protrudes ever further beyond a peripheral surface of the cannula tube in the direction of the tracheal end of the cannula tube. In an embodiment, there is a first and/or a second tube, which are respectively arranged in the first and/or second tube guide of the cannula tube. The depth of the tube guide and/or the diameter of the tubes can vary over their respective length in radial direction of the cannula tube. The first and/or the second tube protrudes ever further out from the tube guide in extension direction. In the direction of the tracheal end, the first and/or second tube rises further out of the tube guide or respectively over the peripheral surface of the cannula tube than at the distal end. The first and/or second tube rises over its length constantly over the peripheral surface of the cannula tube. Advantageously, through the constant rising of the tube over the peripheral surface, a traumatising of the tissue is avoided on withdrawing the tracheal cannula from the trachea.

In a configuration, provision is made that at least the first tube guide and/or the second tube guide is formed in a region of the material buildup of the wall. In a configuration, at least one, and in some embodiments both, tube guides are arranged in a region of the tracheal cannula, which has a material buildup. The tracheal cannula includes two material buildups, lying approximately opposite, which respectively include a tube guide. The at least one material buildup includes a groove which functions as first or second tube guide. The at least one material buildup extends in longitudinal direction of the tracheal cannula. In the region of the tracheal end of the first and/or second tube guide, the tracheal cannula has respectively a material buildup which protrudes substantially over the wall thickness of the surrounded region. A wall thickness between the tracheal end and an end of the first and second tube guide is configured thinner than the wall thickness in the region of the tube guide and has a smaller or no material buildup. The wall thickness with regard to the tracheal cannula increases constantly towards the distal end. The wall thickness in the region of the material buildup in the direction of the longitudinal extent of the tracheal cannula remains the same over the length of the tube guide. In a further embodiment, the wall thickness increases in the region of the material buildup in the direction of the longitudinal extent of the tracheal cannula in the distal direction at least over the length of the tube guide. Advantageously, the first and/or second tube guides can be embedded in an equally deep manner in the material buildup over the longitudinal extent of the tracheal cannula.

In a configuration, provision is made that the wall of the cannula tube has a first external diameter. A portion of the wall, in some embodiments, a portion of the cannula tube, further in some embodiments, the tracheal end portion of the cannula tube, has the first external diameter. The cannula tube, in some embodiments, the tracheal end portion of the cannula tube has the first external diameter at an end facing the cannula shield. The first external diameter is the maximum external diameter of the cannula tube, such as the maximum external diameter of the tracheal end portion of the cannula tube.

In a further configuration, the cannula tube includes a second external diameter. A portion of the wall, in some embodiments, a portion of the cannula tube, and/or the tracheal end portion of the cannula tube, includes the second external diameter. The cannula tube, such as, the tracheal end portion of the cannula tube has the second external diameter in the region of the tracheal end, such as at the tracheal end and/or at a cuff.

The tracheal cannula includes a cannula lumen, continuous from the distal end to the tracheal end, with a constant internal diameter. Further, the first external diameter is greater than the second external diameter. Further, a peripheral surface of the wall is formed in a conical manner. In a configuration, provision is made that the wall of the cannula tube, at least in a tracheal end portion, narrows towards a tracheal end. In a configuration, provision is made that the wall includes a first external diameter, which is assigned to the cannula shield, and a second external diameter, which is assigned to the cuff or respectively to the tracheal end, wherein the first external diameter is greater than the second external diameter. In a further configuration, provision is made that a wall at least of the tracheal end portion includes a first wall thickness contiguous with the distal end portion, and a second wall thickness contiguous with the tracheal end, wherein the first wall thickness is greater than the second wall thickness. In a further configuration, provision is made that a wall thickness of the tracheal end portion reduces continuously or discontinuously towards the tracheal end.

In a configuration, the second external diameter is the smallest external diameter of the tracheal cannula. Further, the second external diameter is the external diameter of the tracheal cannula which is selected by an operator on inserting the tracheal cannula depending on the size of the tracheostoma. The first external diameter, in some embodiments, is approximately 1 mm. In other embodiments, precisely 1 mm greater than a standard size designation of the tracheal cannula, which designates a nominal width, i.e. an internal diameter of the tracheal cannula. Advantageously, an operator does not need to measure an external diameter of the tracheal cannula, but rather can select a proposed tracheal cannula with the aid of the standard size designation for the respectively opened tracheostoma. Here, he only needs to take into account that the first external diameter of the tracheal cannula is approximately 1 mm greater than the standard size designation.

In a configuration, provision is made that the tracheal end portion of the wall, assigned to the tracheal end, is thinner than the distal end portion of the wall, assigned to the cannula shield. In a configuration, provision is made that a first wall thickness of the wall is greater at the site of the first external diameter of the cannula tube than a second wall thickness of the wall at the site of the second external diameter of the cannula tube, wherein the second external diameter is closer to the tracheal end than the first external diameter. The tracheal end portion is configured narrowing tracheally, therefore towards the tracheal end. Further, a portion of the tracheal end portion, assigned to the cannula shield, is configured to be more thick-walled than a portion of the tracheal end portion assigned to the tracheal end. In a configuration, a wall thickness of the wall at the tracheal end portion or respectively at the second external diameter in some embodiments is approximately 0.4 mm to approximately 0.6 mm, and in other embodiments, may be approximately 0.5 mm.

Advantageously, the first and/or second tube at least partially does not protrude over a first and/or second external diameter of the peripheral surface of the cannula tube.

In a configuration, provision is made that the tracheal cannula includes at least one material selected from a group at least including silicone, thermoplastic elastomers, polyvinylchloride and/or polycarbonate.

In a configuration, the cannula tube includes the tracheal end portion and a distal end portion. The distal end portion is assigned to the cannula shield. Further, the distal end portion is able to be arranged outside the trachea. Further, the tracheal end portion is able to be arranged in the trachea. Further, the distal end portion and tracheal end portion differ from one another geometrically. In a configuration, provision is made that the distal end portion is configured substantially in a box-shaped or respectively cubic manner. For example, the distal end portion is configured substantially in a box-shaped manner, wherein the tracheal end portion is configured substantially in a tube-shaped manner. Further, provision is made that the distal end portion is contiguous with a distal side of the tracheal end portion. In an embodiment, the distal end portion is configured in two parts, wherein a first part is connected with the tracheal end portion in an integral manner with regard to material. The second part is connected to the first part in a form-fitting manner and/or in an integral manner with regard to material.

In a configuration, the distal end portion includes a shaping from proximal in the distal direction. In a further configuration, the distal end portion has a lateral shaping, greater than a lateral shaping of the tracheal end portion. In a further configuration, the distal end portion includes a shaping from inferior to superior, greater than a shaping of the tracheal end portion from inferior to superior. In a configuration, provision is made that an extent of the distal end portion from superior to inferior corresponds substantially to an extent of the tracheal end portion at a contact point with the distal end portion from superior to inferior. In a configuration, provision is made that the distal end portion of the cannula tube extends laterally, inferior and/or superior beyond a first external diameter of the tracheal end portion.

The distal end portion includes a superior face, which is delimited by an edge. The superior face is configured in a substantially flat manner.

Further, the superior face is configured in a substantially rectangular manner, in a view from superior.

The distal end portion includes an inferior face which is delimited by an edge. The inferior face is configured substantially in a flat manner. Further, the inferior face is configured in a substantially rectangular manner, in a view from inferior. The inferior face is substantially parallel to the superior face.

The distal end portion includes lateral faces. The lateral faces are configured in a flat, curved, rounded manner, or in a manner which is convex in the lateral direction. The lateral faces are rectangular, in a view from the lateral direction. The lateral faces are delimited by the superior face and the inferior face and by proximal and distal faces.

The distal end portion includes a distal face. On usage of the tracheal cannula, the distal face is substantially parallel to a frontal plane of the user. The distal face is delimited by the lateral faces and the superior and inferior face. The distal face is configured in a flat manner. Further, the distal face includes at least one opening. In a configuration, provision is made that at least the distal end portion has an opening which opens into the tracheal end portion. The opening opens into the cannula lumen.

In a configuration, provision is made that the distal end portion, proceeding from an edge of the opening, extends by approximately 1 mm to approximately 5 mm towards superior and/or inferior. In other embodiments, approximately 1 mm to approximately 2 mm towards superior and/or inferior.

In a configuration, provision is made that the distal end portion has at least a first tube recess for the first tube. The tube recess is configured in a round, lenticular or oval manner in a view from the distal direction. The first tube recess is adapted to a cross-section of the first tube.

The tube recess includes a lenticular, round or oval cross-section. “Lenticular” in the sense of the present disclosure is to be understood to mean a shape which is configured in a convex manner on one side or on both sides. The shape can be flat on one side. Further, the two halves of the lenticular shape can have different radii. In a configuration, the lenticular shape is composed in cross-section from two segments of a circle. The lenticular shape or the oval shape includes an axis of the lenticular shape and two extreme points, which is defined by the contact points of the radii of the individual halves of the lenticular shape or respectively halves of the oval. The axis of the lenticular shape, on use of the tracheal cannula, is substantially parallel to the median plane of the user.

The first tube recess is arranged in a paramedian manner, i.e. distinctly outside the centre axis of the tracheal cannula. Further, the first tube recess is arranged laterally of a connection device for connecting to an inner cannula. Advantageously, the first tube recess is thus situated far enough externally so as to not impede the connection of an inner cannula. In a furthermore advantageous manner, the tube recess is substantially in alignment with the first tube guide of the tracheal end portion. Further, the first tube recess is arranged at a height or inferior with respect to the lateral diameter of the opening. The first tube recess is arranged approximately halfway between lateral diameter of the opening and an inferior edge of the distal face. Advantageously, in such a way a sufficiently large radius of curvature is achieved of the first tube guide or respectively of the first tube, which does not substantially impede a through-flow.

The first tube recess extends into or respectively contiguously up to the first tube guide. Further, the first tube recess extends in a straight or curved manner through the distal end portion.

In a configuration, provision is made that the distal end portion has at least a second tube recess for the second tube. The tube recess is configured in a round, lenticular or oval manner in a view from the distal direction. The second tube recess is adapted to a cross-section of the second tube.

The tube recess includes a lenticular, round or oval cross-section. An axis of the lenticular shape of the second tube recess, in use of the tracheal cannula, is substantially parallel to the median plane of the user.

The second tube recess is arranged in relation to a median plane of the distal end portion lying opposite the first tube recess. The second tube recess is arranged in a paramedian manner, i.e. distinctly outside the centre axis of the tracheal cannula. Further, the second tube recess is arranged laterally of a connection device for connecting to an inner cannula. Advantageously, the second tube recess is thus situated far enough externally so as not to impede the connection of an inner cannula. Furthermore advantageously, the tube recess is substantially in alignment with the second tube guide of the tracheal end portion. Further, the second tube recess is arranged at a height of or inferior with respect to the lateral diameter of the opening. The second tube recess is arranged approximately halfway between lateral diameter of the opening and an inferior edge of the distal face. Advantageously, in such a way a sufficiently large radius of curvature of the second tube guide or respectively of the second tube is achieved, which does not substantially impede a throughflow.

The second tube recess extends into the second tube guide or respectively is contiguous with the second tube guide. Further, the second tube recess extends in a straight or curved manner through the distal end portion.

In a further configuration, provision is made that the distal end portion includes a connection device. The connection device is configured for connection of the tracheal cannula to an inner cannula. The connection device is assigned to the opening. Further, the connection device is arranged at least partially circumferentially around the opening. The connection device is bayonet closure part, which further can interact with a bayonet closure part of an inner cannula. The connection device is arranged on the distal face. Further, the connection device extends from the distal face in the distal direction.

Furthermore, a tube is proposed for use with a described tracheal cannula. In a configuration, provision is made that the tube is configured in an oval, lenticular or circular manner in a cross-section perpendicularly to the longitudinal extent of the first tube. The tube includes a thermoplastic material.

In a configuration, the tube is configured in an oval or lenticular manner. The tube is manufactured from a round tube which is separated apart lengthwise and joined together again after a thermal deformation. In a configuration of the first tube and/or of the second tube, the latter is configured in a lenticular manner in a cross-section. The cross-section of the tube, such as the cross-section of the peripheral surface of the tube, is configured in a lenticular manner. And may be further formed in a convex manner on both sides.

Furthermore, a tube suitable for use with a tracheal cannula, including two convex tube portions in cross-section perpendicularly to longitudinal the extent, extending in longitudinal extent of the first and/or second tube, is proposed, wherein a first tube portion includes in cross-section a first radius R1, and a second tube portion includes in cross-section a second radius R2, wherein the first radius R1 is smaller than the second radius R2.

In a configuration, the tube is the first and/or the second tube, which is described above.

In a configuration, the tube has two tube portions. The tube portions are convex portions, extending in longitudinal extent of the tube, in cross-section or respectively in a view onto an end face. The two tube portions are connected lengthwise, such as materially connected, and without limitation, may be welded. In a configuration, the convex tube portions are formed differently. The first tube portion includes in cross-section a first radius R1. The second tube portion p includes in cross-section a second radius R2. The first radius R1 is smaller than the second radius R2. The first tube portion is assigned to the groove base of the respective tube recess. In a configuration, the outer circumference of the first and/or second tube portion has approximately the same radius as the outer circumference of the cannula tube, such as at the boundary to the distal end portion.

In a configuration, the outer circumference of the first and/or second tube portion has approximately the same radius as a material buildup of the cannula tube, such as approximately the same radius as a material buildup for the tube recess of which the tube is provided. In a configuration, provision is made that the tube is manufactured from a round tube, which is separated apart lengthwise and is joined together again after a thermal deformation.

In a configuration, the tracheal cannula includes a first tube. The first tube can be used as a suction tube. In a configuration, provision is made that the first tube is a suction tube which ends superior to the cuff. In a configuration, provision is made that the first tube is arranged at least partially in the first tube guide. The first tube extends from distally through the first tube recess into the first tube guide. Further, the first tube extends from the distal end portion in the distal direction and has further a connector for a suction device. In a configuration, provision is made that the first tube is configured in an oval, lenticular or circular manner in cross-section. The first tube is configured in a lenticular manner in cross-section. The first tube is adapted to the first tube guide in such a way that there is substantially no joint between the peripheral surface of the tracheal end portion and the first tube. Advantageously hereby an atraumatic, i.e. tissue-conserving, inserting of the tracheal cannula and a comfortable usage is achieved. The first tube is arranged in the first tube guide in such a way that a partial surface of the first tube, protruding out from the first tube guide, supplements an encasing of the peripheral surface. In a configuration, provision is made that the encasing, which encases the peripheral surface and the first tube in the first tube guide is substantially circular or oval in a cross-section. In a configuration, provision is made that an encasing, which encases a peripheral surface of the wall and the first tube in the first tube guide, is substantially constant in a cross-section. In a configuration, provision is made that an encasing, which encases a peripheral surface of the wall and the second tube in the second tube guide, is substantially constant in a cross-section. In an embodiment, provision is made that an outer circumference of the first tube protrudes beyond a circular shape or an oval shape of the peripheral surface or respectively forms an elevation in a circular shape or an oval shape of the encasing. The outer circumference of the first tube protrudes by approximately 0.01 mm to approximately 1 mm beyond a circular shape or an oval shape. The first tube and peripheral surface form a constant surface.

In a configuration, provision is made that the external diameter of the first tube in radial extent of the cannula tube is smaller or approximately equal with respect to a depth of the first tube guide.

In a configuration, the tracheal cannula includes a second tube. The second tube can be used as a filling tube. In a configuration, provision is made that the second tube is a filling tube for the cuff, which extends into the cuff. Air is able to be filled into the cuff by the second tube. In a configuration, provision is made that the second tube is arranged at least partially in the second tube guide. The second tube extends from distally through the second tube recess into the second tube guide. Further, the second tube extends from the distal end portion in the distal direction and has a connector for a suction device. In a configuration, provision is made that the second tube is configured in an oval, lenticular or circular manner in cross-section. The second tube is formed substantially in a circular manner in a cross-section. The second tube is adapted to the second tube guide in such a way that there is substantially no joint between the peripheral surface of the tracheal end portion and the second tube. Advantageously, hereby an atraumatic inserting of the tracheal cannula and a comfortable usage is achieved. The second tube is arranged in the second tube guide in such a way that a partial surface of the second tube, protruding from the second tube guide, supplements an encasing of the peripheral surface. In a configuration, provision is made that the encasing, which encases the peripheral surface and the second tube in the second tube guide, is substantially circular or oval in a cross-section. In an embodiment, provision is made that an outer circumference of the second tube protrudes beyond a circular shape or an oval shape of the peripheral surface or respectively forms an elevation in a circular shape or an oval shape of the encasing. The outer circumference of the second tube protrudes by approximately 0.01 mm to approximately 1 mm beyond a circular shape or an oval shape. The second tube and peripheral surface form a constant surface.

In a configuration, provision is made that the external diameter of the second tube is smaller or approximately equal with respect to a depth of the first tube guide in radial extent of the cannula tube.

In a configuration, the tracheal cannula includes a cannula shield. The cannula shield includes a cannula tube cutout. Further, the cannula cutout extends entirely from proximal to distal direction through the cannula shield. In a top view from the distal direction, the cannula tube cutout is rectangular. Further, the cannula tube cutout includes superiorly and/or inferiorly a straight edge, which further are arranged parallel to one another. Further, the cannula tube cutout includes lateral straight edges lying opposite, which are arranged parallel to one another. Further, the cannula tube cutout includes opposite, curved lateral edges, which are curved in a convex manner in the lateral direction. The cannula tube cutout is delimited at least by a first lateral portion, a second lateral portion and further by a first superior portion and a first inferior portion. The cannula tube is suitable for receiving the distal end portion of the cannula tube. The cannula tube is mounted by a pivot bearing in the cannula tube cutout.

In a configuration, provision is made that the cannula shield has lateral portions delimiting the cannula tube cutout, wherein the pivot bearing is arranged between the lateral portions of the cannula tube cutout and lateral faces of the end portion of the cannula tube. In an embodiment, provision is made that the lateral portions have bearing recesses or bearing pins, which cooperate with bearing pins or bearing recesses which are arranged at the distal end portion. Bearing pins are arranged on the lateral faces of the end portion, or bearing recesses are introduced. A bearing axis about which the cannula shield and cannula tube are able to be tilted about each other, extends in lateral direction. Further, in a top view from distally, the bearing axis is identical with the axis of the diameter of the opening of the end portion. Advantageously, the cannula shield is tiltable relative to the cannula tube, about the lateral axis. Hereby, a comfortable adaptation is achieved of the angle of the cannula shield, which is able to be arranged on a neck surface, and the cannula tube, which is able to be arranged in the trachea. The configuration of the straight posterior and inferior edges has been surprisingly found to be particularly advantageous. Through this configuration, the cannula shield is tiltable with respect to the cannula tube in a greater angle than is known from the prior art. In a configuration, provision is made that through a tilting movement at least one inferior and/or superior gap is at least partially closed or reduced between the distal end portion of the cannula tube and the cannula shield. A gap is reduced between the walls of the cannula tube cutout and the faces of the end portion. Further, the tilting movement is delimited. The tilting movement is delimited in that the cannula shield, a posterior portion, or an inferior portion of the cannula tube cutout abuts against the cannula tube.

In a configuration, the tracheal cannula includes an inner cannula. The inner cannula is configured in a fenestrated or unfenestrated manner. The inner cannula is able to be fastened to the tracheal cannula by a bayonet closure.

In a configuration, the tracheal cannula includes a cuff. The cuff is assigned to the tracheal end portion. Further, the second tube projects into the cuff. Further, the cuff is inflatable by the second tube and/or the air can be extracted from the cuff by this.

In an exemplary embodiment, the tracheal cannula includes a first tube for a subglottic suction, and a second tube for the filling of a cuff. The cannula tube includes on a distal side an opening, around the edge of which a connection device is arranged, in which an inner cannula is able to be connected. Furthermore, the cannula tube includes, by way of example, a screening in the tracheal end portion. A distal end portion of the cannula tube is arranged in a cannula tube cutout and is mounted there in a tiltable manner.

Laterally of the exemplary inner cannula, the first tube is arranged for the filling of the cuff in the distal end portion of the cannula tube. The exemplary inner cannula is fastened to the cannula tube by the connection device. The inner cannula includes a fenestration, which is arranged in the region of the screening of the tracheal cannula.

The distal end portion of the exemplary cannula tube is arranged in the cannula tube cutout and is mounted there in a tiltable manner. The exemplary box-shaped, distal end portion includes a superior face and an inferior face, arranged lying opposite thereto. The exemplary cannula shield has a cannula tube cutout, which is continuous from a distal side to a proximal side and into which the distal end portion is able to be arranged. The cannula shield includes, by way of example, a rectilinear first portion, delimiting the cannula tube cutout, which first portion is assigned to the superior face of the distal end portion. Furthermore, the cannula shield includes, by way of example, a rectilinear second portion, delimiting the cannula tube cutout, which second portion is assigned to the inferior face of the distal end portion. The cannula shield is, by way of example, arranged in a tiltable manner about a medial-lateral axis at the distal end portion by a pivot bearing. The exemplary cannula shield includes lateral portions, delimiting the cannula tube cutout, wherein the pivot bearing is arranged between the lateral portions of the cannula tube cutout and lateral faces of the end portion. On tilting of distal end portion and cannula shield with respect to one another, a gap between these is reduced.

The exemplary cannula tube is divided into a distal end portion and a tracheal end portion, which are contiguous with one another at a contact point. The distal end portion includes, by way of example, the connection device for the inner cannula. The exemplary tracheal cannula includes a cannula lumen, which is continuous from the distal end to the tracheal end, with a constant internal diameter. The cannula lumen is surrounded, by way of example, by a wall which forms the cannula tube, which has a first external diameter which is assigned to the cannula shield. The wall or respectively the cannula tube includes, by way of example, a second external diameter, which is assigned to the tracheal end. The first external diameter is greater than the second external diameter, and a peripheral surface of the wall extends in a conical manner from the first external diameter to the second external diameter. By way of example, a first wall thickness of the wall at the site of the first external diameter is greater than a second wall thickness of the wall at the site of the second external diameter. As the internal diameter is configured in a constant manner, the tracheal end portion narrows in a conical manner in tracheal direction. The wall thickness of the superior and inferior region of the cannula tube, or respectively of the tracheal end portion, reduces continuously from distally in the tracheal direction. Likewise, the wall thickness of the lateral regions of the cannula tube, or respectively of the tracheal end portion, reduces continuously from distally in the tracheal direction. The distal end portion of the cannula tube extends, by way of example, from inferior to superior beyond a first external diameter of the tracheal end portion.

By way of example, the distal end portion is configured in two parts, wherein a first part, by way of example, is connected to the tracheal end portion integrally with regard to material. The second part can be connected to the first part in a form-fitting manner and/or materially. The wall has, by way of example, a first tube guide and a second tube guide, which is arranged on the opposite side of the cannula tube. The first tube is arranged in the first tube guide, and the second tube is arranged in the second tube guide. The first tube and/or the second tube are configured, by way of example, in a lenticular manner in a cross-section, wherein both halves of the lenticular shape have different radii. The first tube guide opens into a first tube recess of the distal end portion. The second tube guide opens into a second tube recess of the distal end portion. An exemplary encasing, which by way of example lies directly against the wall, which encases a peripheral surface of the wall and the first tube in the first tube guide, and the second tube in the second tube guide, is substantially constant. Between the peripheral surface and the first and second tube by way of example there is substantially no joint and no gap.

By way of example, the two tube guides are arranged in a region of the tracheal cannula which has a material buildup. In the region of the tracheal end of the respective tube guide, the tracheal cannula has respectively a material buildup, which protrudes substantially over the wall thickness of the surrounded region. The tube guides are arranged in the material buildup. A wall thickness in the surrounded region of the material buildups of the first and second tube guide is configured thinner in the region of the tracheal end of the tube guides. The wall thickness with regard to the exemplary tracheal cannula increases constantly towards the distal end. The wall thickness in the region of the material buildup, on the other hand, remains the same in the direction of the longitudinal extent of the tracheal cannula. Hereby, the exemplary tube guides can be embedded in an equally deep manner in the material over the longitudinal extent of the tracheal cannula. In a configuration, provision is made that the material buildup has at least partially a radius, such as a radius of the peripheral surface, which is approximately identical to the radius of the cannula tube for instance at the boundary to the distal end portion.

By way of example, the first tube guide resembles an oval segment in a cross-section. Furthermore, by way of example, the second tube guide is configured in a u-shaped manner in a cross-section perpendicularly to the longitudinal extent of the cannula tube.

In an exemplary configuration of the first tube and/or of the second tube, this is configured in a lenticular manner in a cross-section. By way of example, the cross-section of the tube, such as the cross-section of the peripheral surface of the tube, is formed in a convex manner on both sides. By way of example, the tube has two tube portions through a division in extent direction, which tube portions are respectively formed in a convex manner cross-section and are connected lengthwise. By way of example, the convex tube portions are shaped differently. The first tube portion includes by way of example in cross-section a first radius R1, and the second tube portion includes in cross-section a second radius R2, wherein by way of example the first radius R1 is smaller than the second radius R2. The outer circumference of the second tube portion has approximately the same radius R2 as the outer circumference of the cannula tube, for example at the boundary to the distal end portion.

In a configuration, provision is made that an obturator for a tracheal cannula is provided, includes a guide bar, an atraumatic tracheal tip, a distal grip piece, and a sleeve, wherein the grip piece is arranged at a distal end of the guide bar, wherein a portion of the grip piece, facing the guide bar, includes a thread onto which the sleeve is able to be screwed.

By the obturator, an atraumatic inserting of the tracheal cannula into the trachea of a user is advantageously possible. Advantageously, the obturator is adapted to a tracheal cannula. At least the tip of the obturator, such as a maximum diameter of the tip, is adapted to an internal diameter and/or an external diameter of the cannula tube. The tip is able to be arranged in a clamping manner at least in the tracheal end portion of the tracheal cannula. In a configuration, provision is made that the tip includes a rubber-elastic material. For example, the tip includes a maximum diameter which is approximately equal to or greater than an internal diameter of the cannula tube, for which the obturator is provided. Advantageously, the obturator is able to be withdrawn at least partially out from the cannula tube by the sleeve. In a configuration, provision is made that the sleeve is able to be moved by rotation about its longitudinal axis in a translatory manner on the grip piece. When introduced into a tracheal cannula, the sleeve can exert a force against the tracheal cannula, in order to move the obturator and tracheal cannula relative to one another. The force acts on the cannula tube, such as onto the distal end portion of the cannula tube. Further, the force acts exclusively on the cannula tube and may not act on the cannula shield. Advantageously, it is achieved hereby that the cannula shield and cannula tube do not tilt to one another when the obturator is drawn out from the cannula tube.

The guide bar and grip piece are configured in one piece or respectively in a monolithic manner. Further, the tip is held in an engaged manner onto the guide bar or respectively in a form-fitting manner. Further, the sleeve is arranged with play on the grip piece. In a configuration, provision is made that the tip has a maximum tip diameter which is greater than a maximum bar diameter of the guide bar.

In a configuration, provision is made that a bayonet closure part is arranged on the obturator, such as on the grip piece. The bayonet closure part is able to be connected to a bayonet closure part of the tracheal cannula.

In a configuration, provision is made that the thread on the grip piece is partially interrupted. Hereby, as little friction as possible is produced between sleeve and thread. In some embodiments, the per thread pitch may be approximately two to approximately four, and in some embodiments, precisely two thread portions are arranged on the grip piece. The thread portions complement each other or respectively are arranged on the grip piece in such a way that an internal thread of the sleeve is able to be screwed onto these. The grip piece is configured in a substantially smooth manner between the thread portions. In a configuration, provision is made that the thread is a flat thread, a trapezoidal thread or a round thread.

In a configuration, provision is made that the obturator has a bayonet closure part assigned to the grip piece. The bayonet closure part is able to be coupled with the bayonet closure part of the tracheal such cannula, as the cannula shield. Advantageously, the obturator can be held in such a way securely and in a fixed manner in the tracheal cannula. The bayonet closure is detachable from obturator and tracheal cannula, in order to remove the obturator from the tracheal cannula.

In a configuration, the obturator includes a distal plate. The distal plate is arranged at a distal end of the guide bar. The distal plate is formed in a monolithic manner with the guide bar. The distal plate advantageously prevents too wide an introducing of the obturator into a tracheal cannula. The grip piece is able to be fastened on the distal plate.

In an exemplary configuration of the obturator for the atraumatic insertion of the tracheal cannula, the obturator includes a guide bar, an atraumatic tracheal tip of a rubber-elastic material, and a distal grip piece. The grip piece is arranged, by way of example, at a distal end of the guide bar. A portion of the grip piece facing the guide bar includes, by way of example, a thread onto which a sleeve is able to be screwed. The thread is configured, by way of example, as a flat thread and is only partially embodied or respectively is partially interrupted. The tip is connected, by way of example, with the guide bar in a form-fitting manner. The tip has, by way of example, a maximum tip diameter which is greater than a maximum bar diameter.

In a further exemplary configuration, the obturator includes a guide bar, an atraumatic tracheal tip of a rubber-elastic material, and a distal plate. The distal plate is arranged at a distal end of the guide bar and is formed in a monolithic manner therewith.

In a configuration, a set is provided, including at least a tracheal cannula, such as a tracheal cannula described above, and an above-mentioned obturator, wherein a length of the guide bar of the obturator is adapted to a length of the cannula tube of the tracheal cannula. A set of tracheal cannula and obturator is advantageously adjusted to one another, wherein for example a maximum tip diameter and/or an external diameter of the guide bar is smaller than or equal to a maximum internal diameter of the cannula tube. Furthermore, a radius of curvature of the guide bar is approximately equal to a radius of curvature of the cannula tube.

In a configuration, provision is made that tip of the obturator has an external diameter or respectively maximum tip diameter, which is greater than an internal diameter of the cannula tube at the tracheal end. In a configuration, provision is made that the tip of the obturator has a maximum tip diameter, which is approximately identical to or greater with respect to at least an internal diameter of a cannula tube, and may be substantially equal to or greater than an external diameter of the tracheal cannula at the tracheal end thereof. The tip of the obturator or respectively its surface in the inserted state is flush with the cannula tube or respectively its peripheral surface.

The obturator is able to be inserted into the tracheal cannula in such a way that the tip protrudes at least partially at the tracheal end of the tracheal cannula. Further, the tip is adapted to the tracheal end of the tracheal cannula in such a way that it has a maximum diameter which is approximately 0.1 mm to approximately 1 mm greater than the internal diameter of the tracheal cannula. Further, the maximum diameter of the tip is approximately equal to an external diameter of the tracheal cannula at the tracheal end. Advantageously, the tracheal cannula with inserted obturator can be inserted in such a way atraumatically into the tracheostoma.

The tip of the obturator clamps or engages in the tracheal end portion of the tracheal cannula. The rubber-elastic material of the tip relaxes on exit from the distal end, so that a form fit of obturator and tracheal cannula is provided. A surface of the tip is contiguous with a peripheral surface of the tracheal cannula. The tip does not overlap with the peripheral surface. In a configuration, provision is made that with the obturator introduced into the tracheal cannula, by screwing the sleeve on the grip piece, the sleeve is able to exert a force against a distal end of the tracheal cannula. By the force, the tip can be drawn into the tracheal cannula, wherein the tip becomes deformed. Hereby, the removing of the obturator becomes more secure and more comfortable than when the latter is drawn manually out from the tracheal cannula.

In a configuration, the set includes an inner cannula. The inner cannula is removed from the tracheal cannula when the tracheal cannula is placed by the obturator.

In an exemplary configuration of the set, this includes the obturator and the tracheal cannula. The sleeve is screwed onto the thread of the grip piece, by way of example, which sleeve in the assembled state is contiguous with the distal end portion of the tracheal cannula. The guide bar projects, by way of example, through the cannula tube, wherein the tip protrudes at the tracheal end of the tracheal cannula. The maximum tip diameter by way of example is approximately equal to an internal diameter of the cannula tube at the tracheal end.

In a configuration, a method is provided for withdrawing an obturator, described above, from a tracheal cannula, including the steps:
screwing a sleeve on a grip piece of the obturator against the tracheal cannula,
screwing the sleeve against the resistance of the tracheal cannula, whereby at least the guide bar is moved in the distal direction,
withdrawing the obturator from the tracheal cannula.

In a configuration, provision is made that the obturator is withdrawn from the tracheal cannula as soon as the tip is drawn at least by means of screwing the sleeve partially into the tracheal cannula.

Furthermore, the problem is solved according to the present disclosure by a use of an above-mentioned tracheal cannula for the atraumatic placing of the tracheal cannula in a trachea. In a configuration, provision is made that at least a tube guide is used for receiving a tube.

In a configuration, a use at least of an above-mentioned tracheal cannula is provided for receiving a first tube in such a way that an external diameter of the first tube in radial extent of a cannula tube is smaller than or approximately equal to a depth of a first tube guide, or the first tube at least partially supplements a peripheral surface of a cannula tube in such a way that an external circumference of the peripheral surface of the cannula tube is guided further, wherein the external diameter of the first tube has a substantially equal, greater or smaller radius with respect to the peripheral surface in the region of the first tube guide. In an embodiment, provision is made that the first tube in the tube guide is used for the formation of an encasing, substantially constant in a cross-section, of a peripheral surface of a wall of a cannula tube of the tracheal cannula and the first tube.

In a configuration, a use is provided of an above-mentioned tracheal cannula with a cannula shield with a cannula tube cutout, which extends from proximal in the distal direction, wherein the cannula shield has respectively superiorly and inferiorly a rectilinear portion, delimiting the cannula tube cutout, and wherein the cannula shield includes a pivot bearing, for the tiltable arrangement at a distal end portion of a cannula tube, which has a superior face and an inferior face lying opposite thereto. An advantage of the use of the cannula shield is that the freedom of movement of a patient, such as when nodding the head, is increased. This contributes substantially to the comfort of the tracheal cannula in daily use.

### BRIEF DESCRIPTION OF THE DRAWINGS

Further advantageous configurations will emerge from the following drawings. The further developments which are illustrated there are, however, not to be construed as restrictive; rather, the features described there can be combined with one another and with the above-described features to form further configurations. Furthermore, it is to be pointed out that the reference numbers indicated in the description of the figures do not restrict the scope of protection of the present disclosure, but rather only refer to the example embodiments shown in the figures. The same parts, or parts with the same function, have the same reference numbers in the following. There are shown:

FIG. 1 schematically depicts a tracheal cannula;

FIG. 2 schematically depicts a tracheal cannula with an inner cannula;

FIG. 3 schematically depicts a longitudinal section of the tracheal cannula of FIG. 2;

FIG. 4 schematically depicts an exploded view of a tracheal cannula;

FIG. 5 schematically depicts a side view of the tracheal cannula;

FIG. 6 schematically depicts a sectional view VI-VI of FIG. 5;

FIG. 7 schematically depicts a sectional view VII-VII of FIG. 5;

FIG. 8 schematically depicts a sectional view VIII-VIII of FIG. 5;

FIG. 9 schematically depicts a cannula shield with a part of an end portion of the tracheal cannula;

FIG. 10 schematically depicts an obturator;

FIG. 11 schematically depicts an obturator in a tracheal cannula in a longitudinal section;

FIG. 12 schematically depicts a further configuration of an obturator; and

FIG. 13 schematically depicts a sectional view of the first tube.

### DETAILED DESCRIPTION OF EMBODIMENTS

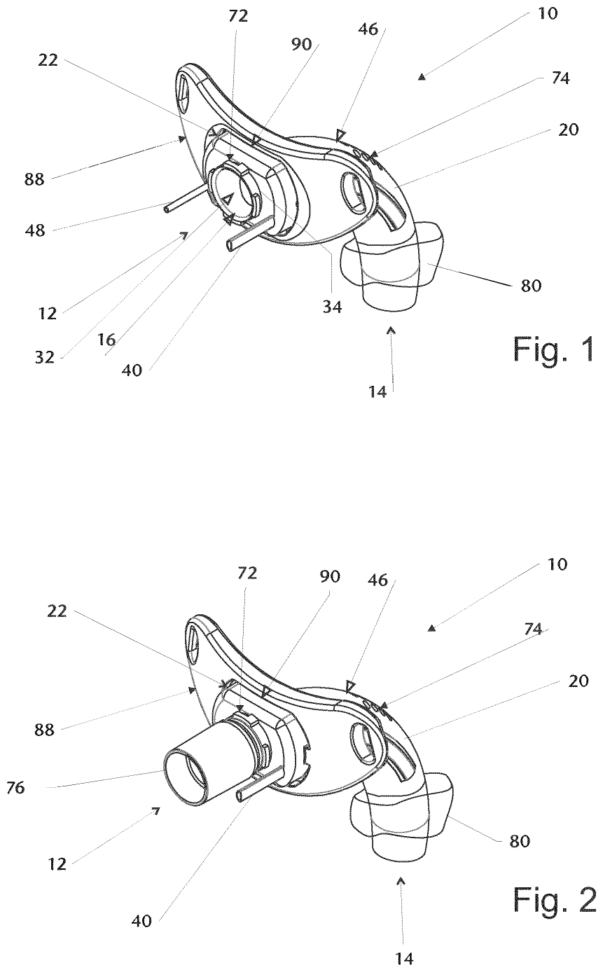

FIG. 1 shows a tracheal cannula 10 with a cannula tube 20 and a cannula shield 88. The tracheal cannula 10 includes a first tube 40 for a subglottic suction, and a second tube 48 for filling a cuff 80. The first tube 40 has a lenticular cross-section. The second tube 48 has a round cross-section. The cannula tube 20 includes on a distal side 12 an opening 32 around the edge 34 of which a connection device 72 is arranged, by means of which an inner cannula, shown in FIG. 2, is able to be connected. Furthermore, the cannula tube 20 includes a screening 74 in the tracheal end portion 46. A distal end portion 22 of the cannula tube 20 is arranged in a cannula tube cutout 90 and is mounted in a tiltable manner there.

FIG. 2 shows the tracheal cannula 10 of FIG. 1 with the cannula tube 20 and the cannula shield 88 and an inner cannula 76. Laterally of the inner cannula 76 in the distal end portion 22 of the cannula tube 20, the first tube 40, which is lenticular in cross-section, is arranged for the filling of a cuff 80. The inner cannula 76 is fastened to the cannula tube 20 by the connection device 72. Furthermore, the cannula tube 20 includes a screening 74 in the tracheal end portion 46. The distal end portion 22 of the cannula tube 20 is arranged in the cannula tube cutout 90 and is mounted in a tiltable manner there.

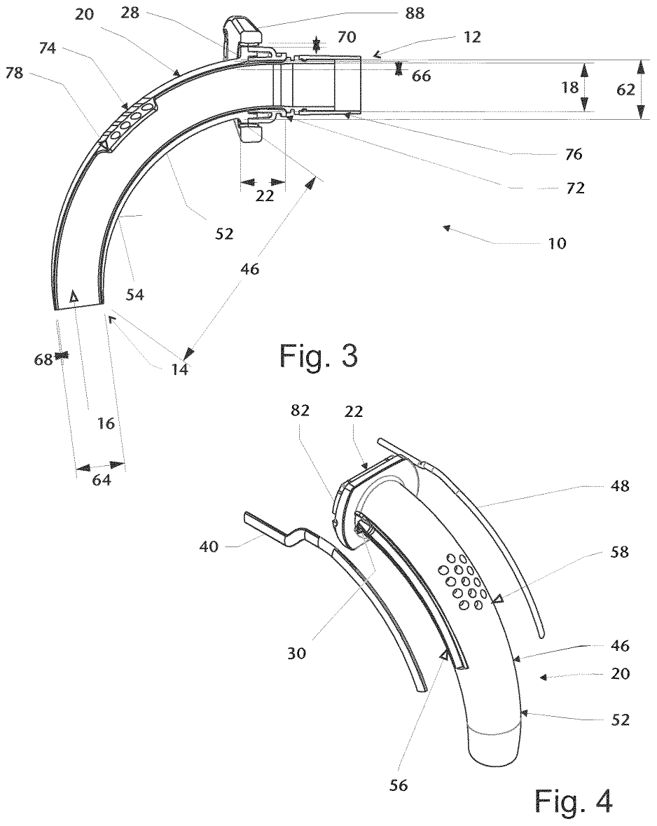

FIG. 3 shows a cross-section of the tracheal cannula 10 of FIG. 2. The cannula tube 20 is divided into a distal end portion 22 and a tracheal end portion 46, which are contiguous with one another at a contact point 28. The distal end portion 22 is mounted in the cannula shield 88. When the distal end portion 22 and cannula shield 88 tilt with respect to one another, a gap 70 between these is reduced. The distal end portion 22 includes the connection device 72 for the inner cannula 76. The inner cannula 76 has a window 78, which is able to be assigned to the screening 74 of the tracheal cannula 10. The tracheal cannula 10 includes a cannula lumen 16 which is continuous from the distal end 12 to the tracheal end 14, with a constant internal diameter 18. The cannula lumen 16 is surrounded by a wall 52, which forms the cannula tube 20, which wall has a first external diameter 62, which is assigned to the cannula shield 88. The wall 52 or respectively the cannula tube 20 includes a second external diameter 64, which is assigned to the tracheal end 14. The first external diameter 62 is greater than the second external diameter 64 and a peripheral surface 54 of the wall 52 extends conically from the first external diameter 62 to the second external diameter 62. A first wall thickness 66 of the wall 52 at the site of the first external diameter 62 is greater than a second wall thickness 68 of the wall 52 at the site of the second external diameter 64. As the internal diameter 18 is configured in a constant manner, the tracheal end portion 46 narrows conically in tracheal direction. The wall thickness 66, 68 of the superior and inferior region, visible here, of the cannula tube 20 or respectively of the tracheal end portion 46, reduces continuously from distally in the tracheal direction. The distal end portion 22 of the cannula tube 20 extends from inferior to superior beyond a first external diameter 62 of the tracheal end portion 46.

FIG. 4 shows a cannula tube 20 with the first tube 40 and with the second tube 48 in an exploded view. The distal end portion 22 is configured in two parts, wherein a first part 82 is connected integrally with regard to material to the tracheal end portion 46. The second part 84, which is shown in FIG. 7, can be connected to the first part 82 in a form-fitting manner and/or materially. The wall 52 has a first tube guide 56 and a second tube guide 58, which is arranged on the opposite side of the cannula tube 20. The first tube 40 is arranged in the first tube guide 56, and the second tube 48 is arranged in the second tube guide 58. The first tube guide 58 opens into a first tube recess 30 of the distal end portion 22.

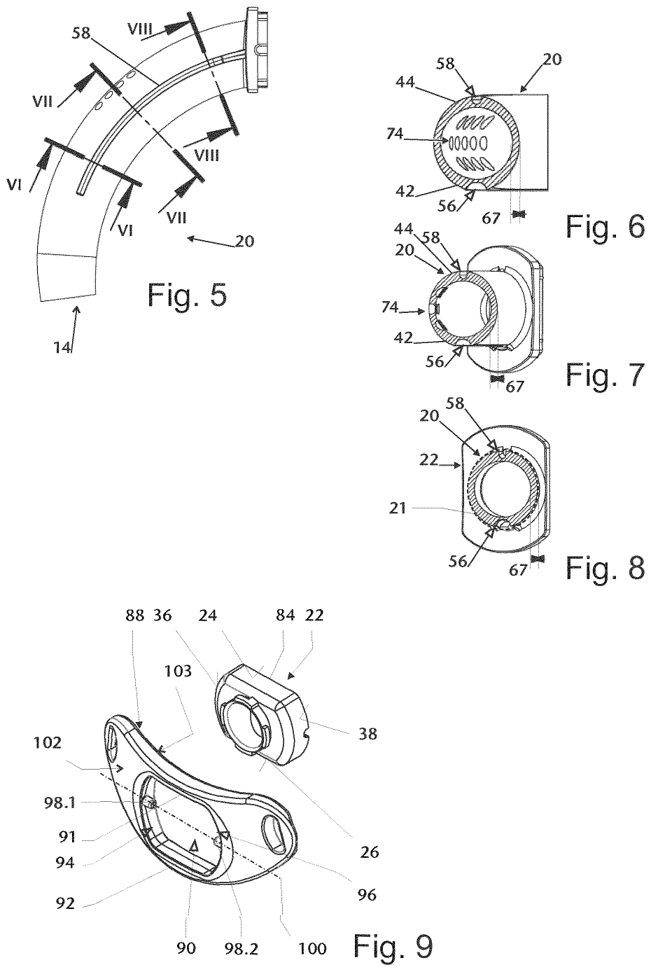

FIG. 5 shows a side view of the cannula tube 20 without tubes 40 and 48. In FIG. 5, the sections VI-VI, VII-VII and VIII-VIII are indicated in different portions of the tube guide 58, which are shown in FIG. 6, FIG. 7 and FIG. 8.

FIG. 6 shows a section VI-VI of the tracheal cannula 20 of FIG. 5 in the region of the tracheal end 14 of the second tube guide 58 viewing in the direction of the screening 74. This has respectively, in the region of the first tube guide 56 and the second tube guide 58, material buildups 42, 44, in which the tube guides 56 and 58 are arranged. A wall thickness 67 is configured thinner in the surrounded region of the material buildups 42, 44 of the first and second tube guide 56 and 58. The cannula lumen 16 is configured in a circular manner.

FIG. 7 shows a section VII-VII of the tracheal cannula 20 of FIG. 5 in the region of the screening 74. Here, also, in the region of the tube guides 56 and 58 a material buildup 42, 44 is arranged which, however, is less intensive in relation to the wall thickness 67, as the wall thickness 67 is greater in the region of the screening than in the region of the tracheal end.

FIG. 8 shows a section VIII-VIII of the tracheal cannula 20 of FIG. 5 at the boundary to the distal end portion 22. The wall thickness 67 is approximately equally thick over the outer circumference 21 of the tracheal cannula 20. As the wall thickness 67 becomes greater towards the distal end portion 22, this is sufficiently thick there in order to receive the tube guides 56 and 58 or respectively the tubes which are not shown here.

In FIG. 6, FIG. 7 and FIG. 8 it can be seen that the first tube guide 56 has a cross-section which resembles an oval segment. Furthermore, the second tube guide 58 is configured in a u-shaped manner. Furthermore, it can be seen from FIG. 6, FIG. 7 and FIG. 8 that the wall thickness 67, of the lateral region of the cannula tube 20 or respectively of the tracheal end portion 46, which is designated in FIG. 2 and FIG. 3, reduces continuously from distal direction to tracheal direction.

FIG. 9 shows the cannula shield 88 and a second part 84 of the distal end portion 22 in an exploded view. The box-shaped, distal end portion 22 includes a superior face 24 and an inferior face 26 arranged lying opposite thereto. The cannula shield 88 has a cannula tube cutout 90, which is continuous from a distal side 102 to a proximal side 103 and into which the distal end portion 22 is able to be arranged. The cannula shield 88 includes a rectilinear first portion 91, delimiting the cannula tube cutout 90, which first portion is assigned to the superior face 24 of the distal end portion 22. Furthermore, the cannula shield 88 8 includes a rectilinear second portion 92, delimiting the cannula tube cutout 90, which second portion is assigned to the inferior face 26 of the distal end portion 22. The cannula shield 88 is arranged in a tiltable manner about a medial-lateral axis 100 by a pivot bearing 98.1 and 98.2 at the distal end portion 22. The cannula shield 88 includes lateral portions 94, 96 delimiting the cannula tube cutout 90, wherein the pivot bearing 98 is arranged between the lateral portions 94, 96 of the cannula tube cutout 90 and lateral faces 36, 38 of the end portion 22.

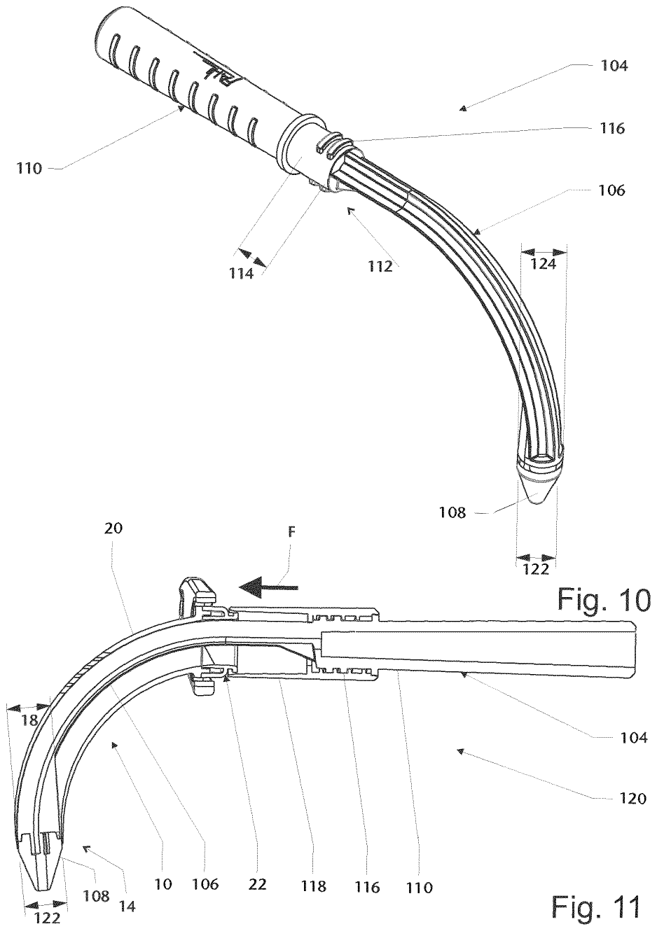

FIG. 10 shows an obturator 104 for the atraumatic insertion of the tracheal cannula 10. This includes a guide bar 106, an atraumatic tracheal tip 108 of a rubber-elastic material, a distal grip piece 110. The grip piece 110 is arranged at a distal end 112 of the guide bar 106, and is formed therewith in a monolithic manner. A portion 114 of the grip piece 110, facing the guide bar 106, includes a thread 116, onto which a sleeve 118 is able to be screwed, which is shown in FIG. 11. The thread 116 is configured as a flat thread and is only partially embodied or respectively is partially interrupted. The tip 108 is connected to the guide bar 106 in a form-fitting manner. The tip 108 has a maximum tip diameter 122 which is greater than a maximum bar diameter 124.

FIG. 11 shows a set 120 including the obturator 104 of FIG. 10, and the tracheal cannula 10 of FIG. 1 in assembled state in a longitudinal section. The sleeve 118 is screwed onto the thread 116 of the grip piece 110, which sleeve is contiguous with the distal end portion 22 of the tracheal cannula 10. The guide bar 106 projects through the cannula tube 20, wherein the tip 108 protrudes out at the tracheal end 14 of the tracheal cannula 10. The maximum tip diameter 122 is approximately equal to an internal diameter 18 of the cannula tube 20 at the tracheal end 14.

The sleeve 118 can be screwed against the distal end portion 22 and can thus exert a force F thereon. Through the force F, tracheal cannula 10 and obturator 104 are moved relative to one another, wherein the tip 108 is drawn back into the cannula tube 20.

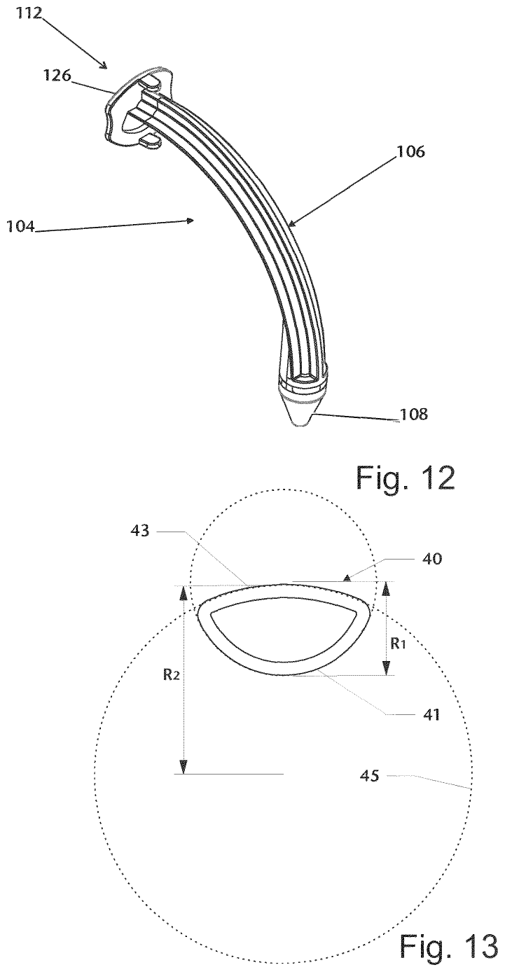

FIG. 12 shows a further configuration of an obturator 104. This includes a guide bar 106, an atraumatic tracheal tip 108 of a rubber-elastic material and a distal plate 126. The distal plate 126 is arranged at a distal end 112 of the guide bar 106 and is formed in a monolithic manner therewith. The distal plate 126 prevents too far an inserting of the obturator 104 into a tracheal cannula 20, which can be seen for example in FIG. 1. The tip 108 is connected to the guide bar 106 in a form-fitting manner. The tip 108 has a maximum tip diameter 122 which is greater than a maximum bar diameter 124.

FIG. 13 shows the first tube 40 in a cross-section. The first tube 40 is configured in a lenticular manner in cross-section, wherein convex tube portions 41, 43 are formed differently. The first tube portion 41 includes in cross-section a first radius R1, and the second tube portion 43 includes in cross-section a second radius R2, wherein the first radius R1 is smaller than the second radius R2. The outer circumference 45 of the second tube portion 45 has approximately the same radius R2 as the outer circumference 21 of the cannula tube 20, which can be seen from FIG. 8, at the boundary to the distal end portion 22.

Advantageously, by the proposed tracheal cannula and the proposed obturator, an inserting of the tracheal cannula can be carried out in a substantially more tissue-conserving manner. The wear comfort is also substantially improved by the proposed tracheal cannula.

1. A tracheal cannula comprising:
a cannula tube; and
a cannula shield, wherein the cannula tube has a distal end portion, to which the cannula shield is assigned, and a tracheal end portion, which is able to be arranged in a trachea, wherein a first external diameter is assigned to the cannula tube at the cannula shield;
a second external diameter is assigned to the cannula tube at a tracheal end,
wherein the cannula tube includes a cannula lumen which is continuous from a distal end to the tracheal end and has a constant internal diameter, wherein a wall at least of the tracheal end portion includes a first wall thickness which is contiguous with the distal end portion, and a second wall thickness at the tracheal end, wherein the first wall thickness of the wall at the site of the first external diameter is greater than a second wall thickness of the wall at the site of the second external diameter, wherein the first wall thickness is greater than the second wall thickness, and the wall thickness of the tracheal end portion reduces continuously towards the tracheal end.

2. The tracheal cannula according to claim 1, wherein the wall of the cannula tube of the at least of the tracheal end portion has at least on one side a material buildup in a cross-section perpendicularly to an extent of the cannula tube.

3. The tracheal cannula according to claim 1, wherein an outer circumference of the tracheal cannula is at least partially substantially oval in a cross-section perpendicularly to an extent of the cannula tube.

4. The tracheal cannula according to claim 1, further comprising:
at least a first tube guide which extends from a distal side of the cannula shield at least up to a cuff, which is assigned to the tracheal end.

5. The tracheal cannula according to claim 4, wherein at least the first tube guide is formed at least partially as a groove in the wall of the tracheal end portion.

6. The tracheal cannula according to claim 4, wherein at least the first tube guide is formed in a thickened region of the wall of the tracheal end portion.

7. The tracheal cannula according to claim 4, wherein a first tube is arranged at least partially in the first tube guide.

8. The tracheal cannula according to claim 7, wherein the first tube is configured in an oval, lenticular or circular manner in a cross-section perpendicularly to an extent of the cannula tube.

9. The tracheal cannula according to claim 7, wherein an external diameter of the first tube is smaller than or approximately equal to a depth of the first tube guide in radial extent of the cannula tube.

10. The tracheal cannula according to claim 4, further comprising:
at least a second tube guide extending from a distal side of the cannula shield at least up to the cuff and in which a second tube is guided.

11. The tracheal cannula according to claim 10, wherein the second tube is a filling tube for the cuff, which extends into the cuff.
